(19) United States
(12) Patent Application Publication
(10) Pub. No.: US 2005/0074880 A1
(43) Pub. Date: Apr. 7, 2005

(54) Generation of multipotent central nervous system stem cells

(76) Inventors: Hoi U Sang, Santa Fe, CA (US); Farid Saliooque, San Diego, CA (US); Warren J Alilain, Detroit, MI (US)

Correspondence Address: FISH & RICHARDSON, PC, 12390 EL CAMINO REAL, SAN DIEGO, CA, 92130-2081, US

(21) Appl. No.: 10/473,003

(22) PCT Filed: Mar. 23, 2002
(86) PCT No.: PCT/US02/09160

Related U.S. Application Data
(60) Provisional application No. 60/278,510, filed on Mar. 23, 2001.

(51) Int. Cl. C12N 15/85; C12N 5/08

(52) U.S. Cl. 435/455; 435/368

(57) ABSTRACT

Methods for generating various cellular phenotypes from central nervous system stem cells are disclosed. Cellular differentiation into phenotypes of organs and tissues within and outside of the central nervous system is induced by co-culture with target cell types or by soluble trophic factors and elements of the extracellular matrix. Established pluripotent CNS stem cell lines are also disclosed.

### BACKGROUND OF THE INVENTION

This application claims priority under 35 U.S.C. § 119(e), to U.S. provisional patent application Ser. No. 60/278,510, filed Mar. 23, 2001.

### FIELD OF THE INVENTION

The present invention generally concerns a method for the in vitro culture and proliferation of pluripotent neural stem cells, and to the use of these cells and their directed progeny as tissue grafts and in cell repopulation. The invention more specifically relates to a method for the isolation and in vitro perpetuation of large numbers of non-tumorigenic neural stem cell progeny which can be induced and directed to differentiate into neuronal and non-neuronal cell types that can be used for repopulation in the undifferentiated or differentiated state to treat disease, degeneration and trauma to the central nervous system (CNS), or potentially any organ or tissue. This invention further relates established CNS pluripotent cell lines and to methods for utilizing the established stem cell lines as research platforms to discover novel factor(s) (e.g., proteins and genes), for generating various differentiated cell types for drug screening, autologous or homologous transplantation, and in vivo proliferation and differentiation of the transplanted stem cell progeny in the host.

### DESCRIPTION OF RELATED ART

Central nervous system (CNS) stem cells give rise to glia and neurons in response to trophic factors (1-3). The development of these cells in the brain may be influenced by local microenvironmental factors. Both fetal and adult progenitor cells give rise to neuronal and glial phenotypes upon implantation into the fetal (4), newborn (5) and adult brain (6, 7). Region specific development has also been observed when CNS stem cells are implanted into neurogenic areas of the adult brain such as the hippocampus where stem cells are found naturally (8).

It is well understood that it would be desirable to develop a well-defined, reproducible source of pluripotent cells available in unlimited amounts for transplantation, drug screening, and for study of function, dysfunction, or development within the various organs and tissues of the body. The instant invention provides both the sources and the methods for developing additional sources of such versatile cells.

### SUMMARY OF THE INVENTION

To address the beforementioned problem and the above solution the inventors disclose their invention as follows.

The instant invention contemplates pluripotent stem cells, for example, mammalian central nervous system (CNS) stem cells isolated from fetal, neonatal or adult brain, as well as resulting cell lines and cell cultures. These cells have the capacity to proliferate perpetually in an undifferentiated state as, for example, CNS stem cells. When these stem cells, for example, CNS stem cells, are grown in or exposed to an environment of cells comprising ectoderm, mesoderm or endoderm tissue cells, or soluble stimulating factors, or media conditioned by such cells or factors, they have the capability to differentiate into functional cells of the ectoderm, mesoderm or endoderm tissue groups.

Co-culturing with other mammalian cell types, or culturing in the presence or absence of soluble factors or signals induces stem cells, for example CNS stem cells to differentiate into neurons, glia and other cell types. For example, in one embodiment the absence of beta Fibroblast Growth factor (bFGF) in their growth medium induces these cells to differentiate into cells with glial and neuronal properties.

In another embodiment, isolated factors or signals from adjacent endocrine cell types induces the isolated stem cells, for example CNS cells to differentiate into endocrine cells that are capable of producing, for example, insulin Thus, the isolated stem cells, for example CNS cells, can be differentiated to become insulin-producing beta cells normally found in the islets of Langerhans cells of the pancreas.

In another embodiment, for example, stem cells, for example CNS stem cells isolated in accordance with the invention described and claimed herein can be induced to differentiate to pituitary cells that have the capability to produce one or more members of the group of pituitary factors consisting of growth hormone, prolactin, and pit1. In another more preferred embodiment, pituitary differentiation is induced by factors or signals isolated from other mammalian pituitary cells, causing the generation of pituitary cells.

In yet another embodiment, the isolated pluripotent stem cells, for example are differentiated into cardiac cell types. Such cardiac cell types include pulsatile cardiac cells, having the capacity to express one, or more, cardiac transcription factors. Preferably, these transcription factors comprise the group consisting of GATA-4, myosin, or troponin IC.

In yet a farther embodiment, the isolated stem cells, for example CNS cells differentiate into glial cell types in the presence of other mammalian cell types. This can be accomplished by exposing stem cells, for example, CNS stem cells to mammalian Post Natal-5 days primary astrocytes culture, mammalian glioma cultures, or isolated factors and/or signals from other mammalian cell types. Differentiation may be confirmed, for example, by analysis for the presence or expression of glial fibrillary acidic protein (GFAP). In another embodiment the stem cells are differentiated into neurons in the presence or absence of factors or signals from other mammalian cell types. Preferably, the cells respond to the presence of epidermal growth factor (EGF) and bFGF by differentiating into neurons expressing microtubule associated protein 2 (Map-2) marker. The cells also respond to the presence of BDNF by differentiating into neurons expressing Map-2 marker.

Also contemplated by the instant invention is a method for inducing trans-differentiation of pluripotent central nervous system stem cells into various other cell types. This method comprises harvesting the pluripotent stem cells from tissues and organs, placing the harvested cells into cell culture, and culturing the cells under conditions suitable for maintaining their pluripotency. Subsequently, the cultured pluripotent cells are contacted with differentiation-inducing factors. Thereafter, differentiation into a particular cell type can be determined, for example, by characterizing the expression of cell-specific properties.

One method for harvesting the cells comprises teasing or trituration of fetal, neonatal or adult CNS tissue, for example, and placing the dissociated cells on poly-L-ornithine coated culture plates. Differentiation is accomplished, for example, by contacting the isolated cells with desired soluble factors, cell-conditioned media, or with co-cultured non-homologous cells, i.e., cells from a desired tissue source. The differentiation inducing cells are typically maintained in standard media, after which the conditioned media may be decanted and added to stem cells in culture, thereby exposing them to soluble stimulants secreted by the inducing cells. Alternately, the contacting can be accomplished by co-culturing with organ-specific inducing cell types, as noted above. Induction of differentiation can also be achieved by exposure to tissue specific factor(s)(e.g., transcriptional factor[s]) or insertion into the stem cells. Determination of stem cell differentiation may be made, for example, by quantitative reverse transcriptase-polymerase chain reaction (QRT-PCR). This determination can also be made, for example, by immunocytochemical characterization of the expression of cell-specific markers. For example, Cell-specific markers that may be used to identify various directed differentiated cells of the present invention include protein molecules such as nestin, MAP-2, GFAP, Insulin, Lhx-3, Pit-1, prolactin, GATA4, myosin and troponin IC. The presence of nestin indicates that the proliferating cells have stem cell properties. MAP-2 indicates differentiation into neuronal cells, whereas GFAP indicates differentiation into glial cells. Transcription factors Lhx-3 and Pit-1 as well as growth hormones hGH and Pr1, indicate differentiation into pituitary cells, and GATA-4, myosin, or troponin IC indicate differentiation into pulsatile cardiac cells. It can be seen, therefore, that the number of differentiated cell types is quite extensive, and may extend to even other, previously uncontemplated cell types.

Further contemplated by this invention is a method for treating diseases involving various CNS and non-CNS organs and tissues of a subject by populating or repopulating cells in, for example, depleted or defective organs or tissues with pluripotent CNS stem cells. Preferably, these cells are induced to differentiate in vivo upon being transplanted into a subject More preferably, they are induced to differentiate in vitro into functional cell types of the target organ or tissues prior to transplanting by placing the harvested pluripotent CNS cells into cell culture and culturing and/or contacting them with, for example, differentiation-inducing cells, cell-conditioned media, and/or factors. Most preferably, after determining the presence of differentiation into a desired cell type, committed progenitor cells are transplanted into a subject to populate or repopulate target tissue or, for example, defective or depleted areas of target tissues and organs. The populating or repopulating can be accomplished, for example, by grafting, gene therapy, factor delivery, tissue engineering and organ development In yet another preferred embodiment, differentiated stem cells, for example, differentiated CNS stem cells can be used as a conduit for gene therapy or for factor delivery to prevent or treat a disease.

Still further contemplated by the invention is a method for identifying functionality of certain genes, proteins and regulation in various organ and tissue cell types. This is useful in gene discovery, drug discovery, elucidation of differentiation pathways, genetic markers, regulatory factors and determination of biological regulation. Most preferably, the differentiated stem cells, for example, differentiated CNS stem cells can be used in vitro or in vivo to produce biological factors such as hormones and other vital proteins.

These and other aspects and attributes of the present invention will become increasingly clear upon reference to the following drawings and accompanying specification.

### BRIEF DESCRIPTION OF THE DRAWINGS

FIG. 1A

FIG. 1B shows (upper) expression of nestin, Map2 and GFAP messages in rat CNS system, and (lower) expression of nestin, Map2 and GFAP proteins in rat CNS stem cells.

FIG. 2 demonstrates nestin expression by RSCs on day 0 (Top Left) and on day 14 after exposure to BGF+□□-FGF (Middle Left).

Also shows M 2 expression by RSCs on day 0 (Top Right), on day 14 after exposure to BGF+□-FGF (Middle Right), and on day 14 after exposure to BDNF (Bottom Right).

FIG. 3a shows RSCs labeled with Bisbenzmide (Bis) prior to co-culture with P5 astrocytes (Row 1 and 2), and adult astrocytes (Row 3 and 4), Bisbenzimde+ cells are therefore RSC derived (Left Column), Bisbenzimide+ cells in the same field are also double-stained for nestin (Row 1 and 3, right). Some Bizbenmide+ cells retained a flattened morphology like stem cells and remain nestin+. Most Bisbenzimide+cells assumed a stellate shape similar to astrocytes in the zP5 co-cultures and expressed GFAP. The number of Bisbenzimide+/GFAP+ cells in the adult co-cares is rare.

FIG. 3b demonstrates cell marker expression in RSC/P5 and RSC/adult astocyte co-cultures.

FIG. 4a shows RSC cultures exposed to DNE/F12+N2+5% FBS culture media (left) or C6 conditioned media (Right). The expression of nestin (Top) and GFAP (bottom) was determined. While the expression of nestin declined, the expression of GFAP (Bottom) was induced. The induced cells assumed an astrocyte-like shape with extension of multiple processes.

FIG. 4b shows cell marker expression in RSC cultures exposed to C6 conditioned media.

FIG. 5 demonstrates In vivo differentiation of RSCs implanted in adult rat brains.

Identification of labeled progenitor cells after inoculation into rat brains. Adult rat brain 4 weeks after inoculation into the periventricular region (Left) LacZ-labeled progenitor cells are observed under the ependyma. (Right) Vibrotome sections (50 um) were evaluated with IM to determine the expression of nestin in the grafted cells. A significant number of cells in the graft were nestin+. Adult rat brain 4 weeks after inoculation into the periventricular region. Vibrotome sections (40 um) were evaluated with IM to determine the expression of MAP-2 and GFAP in the grafted cells. A significant number of cells in the grafts were MAP-2 positive (Left). The number of GFAP+ cells was considerably smaller (Right).

FIG. 6 shows the expression of pit1, prolactin and nestin in rat CNS stem cells and GH₃ cells (top). Induction of Lhx3 and pit1 in rat CNS stem cells by GH₃ conditioned media. (bottom) RSCs were labeled with Bisbenzmide (Bis) prior to co-culture. Bisbenzimide+ cells were therefore RSC derived (ft Column). Bisbenzimide+ cells in the same field were also double-stained for nestin (Row 1, right), Pit-1 (row 2, right), Growth Hormone (GH) (Row 3, right), and Prolactin (Pr1) (Row 4, right). Some Bisbenzimide+ cells retained a flattened morphology like stem cells and remained nestin+. Most Bisbenzimide+ cells assumed a spherical shape similar to GH₃ cells and expressed Pit-1, Growth Hormone and Prolatin

FIG. 7b demonstrates cell marker expression in RSC/GH₃ co-cultures.

FIG. 7c shows expression of nestin, Pit1, Pr1, and growth hormone in Rsc exposed to GH3 conditioned (GH3 CM).RSC cultures were exposed to DME/F12+N2 cults media [—GH₃CM] (left) or Gs conditioned media [+GH₃CM] (Right). The expression of nestin (Row 1), Pit-1 (Row 2), Growth Hormone (Row 3) and Prolactin (Row 4) was determined. While the expression of nestin decline, the expression of Pit-1 (Row2), Growth Hormone (Row 3) and Prolactin (Row4) was induced. The induced cells assumed a spindle shape.

FIG. 7d demonstrates cell marker expression in RSC culture exposed to GH3 conditioned media.

FIG. 8a demonstrates induction of GATA4 and cardiac myosin heavy chain (MHC) in rat CNS stem cells treated with GDNF.

FIG. 8b induction of myosin and troponin IC in RSCs by GDNF is shown RSCs were exposed to GDNF (100 ng/ml) for 20 days. Decrease in nestin (Top) expression is associated with induction of myosin (Middle) and troponin IC (Bottom) expression.

### DESCRIPTION OF THE PREFERRED EMBODIMENT

Introduction

Central nervous system (CNS) stem cells give rise to neurons and glia when exposed to specific trophic factors. In studies with rat fetal brain derived stem cells (RSCs), it has been demonstrated that RSCs can be induced to express the developmentally regulated transcription factors and cell markers characteristic of cells derived from other germ layers, e.g., cardiac myocytes, pancreatic cells and pituitary cells. Therefore, RSCs are not restricted to a defined developmental fate. They may retain pluripotentiality and can be redirected to develop into other cell types not found in the brain provided the correct set of stimuli is present.

In order to characterize these lineage-promoting influences, cultured cells with well-defined phenotypes were studied and found to influence the developmental fate of rat fetal CNS stem cells (RSCs). For example, the influence of one CNS cell type, the astrocyte, on the development of RSCs was investigated by co-culture with either neonatal (P5) astrocytes or transformed tumorigenic C6 glioma cells. Both types of cells stimulated RSCs to assume the morphologic and cell type specific protein expression patterns characteristic of astrocytes. This specific induction effect was also observed in RSCs exposed to media conditioned by C6 cultures suggesting this occurred through the action of secreted factors. Co-culture with adult astrocytes however did not exert any glial inductive effect. In order to determine whether this cell type specific inductive phenomenon was unique to cells of the CNS, these effects were further explored using cells derived from a different germ layer such as the endoderm (9).

RSCs were co-cultured with rat pituitary adenoma GH₃ cells. RSCs exposed to GH₃ cells as well as to GH₃ conditioned media developed the morphologic and protein expression features characteristic of pituitary cells. While not being bound by this mechanism, it is believed that this may have occurred through the induction of Lhx 3 and Pit-1, transcription factors which are essential to pituitary development (10-17). Thus, cells of a different germ layer origin can influence the development of CNS ectoderm derived RSCs. To test whether these trans-germ layer induction effects were due to specific factors, RSCs were also treated with a host of known and well characterized growth/differentiation factors and it was discovered that glia derived growth factor (GDNF) induced RSCs to exhibit rhythmic contractile activities as well as the protein expression patterns characteristic of cardiac myocytes which are of mesodermal origin. The induction of CNS stem cells to acquire cell fates across germ layer boundaries under specific conditions demonstrates that seemingly committed stem cells possess differentiation potentials beyond their organ of origin The development of multiple cell fates under the influence of different and varied conditions also demonstrates that the genesis of cell fate is likely mediated through an instructive rather than a permissive mechanism(s).

### EXAMPLE I

### Isolation and characterization of Human Fetal CNS Stem Cells (HSC′) and Rat Fetal CNS Stem Cells (RSCS)

Isolation and maintenance procedures

Harvesting Cells from Tissue

Human or rat fetal brain tissue was excised from a single or multiple sources and immediately placed into ice-cold Dulbecco's Modified Eagle Medium. The tissue was taken out of media and diced into larger fragments (5-25 mm³). All blood, vascular and connective tissues were removed. Fragments were then placed in Dulbecco's Modified Eagle Medium and diced as small as possible (1-5 mm³) The diced tissue was transferred to a sterile tube where a 1:1 to 1:3 mixture of Dulbecco's Modified Eagle Medium and ATV solution (a premixed 0.5 gm/L trypsin and 0.2 gm/L EDTA*4Na in Hank's buffer, Gibco) was added at 5 to 10 times tissue volume. The tube and content were placed in a 37° C. agitating water bath for 5-15 minutes. Furthermore, the tube was shaken and inverted, by hand, for 5 to 10 seconds once every three to four minutes.

Serum supplemented media may or may not be added at this juncture. This is dependent on the texture and consistency of the tissue. If digest is complete and no visible clumps are present, which is usually the case using tissue from very young rat pups, then serum supplemented media is added to stop further digestive activity. If the digest is not complete, further exposure to ATV solution will continue until the cells are plated in serum supplemented media A 5 ml fire-polished glass pipette or a pipette of equivalent orifice size is then used to further separate tissue by sustained pipetting for a period of 30 to 120 seconds.

Tissue may or may not be filtered If there is a lot of extraneous tissues (e.g. connective, skeletal or vascular) mixed in the brain digest, filtering is used to remove them. Usually other tissues from the head region will not dissociate as-readily as brain. Filtering will also remove larger pieces of any un-disassociated brain tissue. Filter pore size can be crucial, and it has been observed that most stem cell colonies form around cell clusters that have managed to pass through the filtering process.

i) Filter Method

The content of the tube was filtered through a sterilized 60-mesh Nytex membrane and the recovered volume cen ged at 140 to 150 Relative Centrifugal Force units for five to ten minutes.

ii) Filterless Method

The content of the tube was centrifuged at 140 to 150 Relative Centrifugal Force units for five to ten minutes. The liquid phase was removed and the cell pellet resuspended in appropriate volume of Dulbecco's Modified Eagle Medium supplemented with 0% Fetal Bovine Serum (FBS). The serum will stop the ATV solution's digestive activity.

Plating of Cells

The cells were plated onto tissue culture vessels, which have been treated overnight with Poly-L-ornithine at 0.005 to 0.02 mg/cm² (Sigma). Cells were plated at a density of 20,000/cm² to 75,000/cm², preferably on 35 mm to 100 mm diameter plates (Falcon). The newly plated culture was placed in a 37° C. incubator with a CO₂ content of 5.2% for a period of 24 to 72 hours depending on initial cell to plate attachment ratio and subsequent number of surviving cells (35% to 80%). Media was then changed to serum-free defined media consisting of Dulbecco's Modified Eagle Medium/F12 containing N2 supplement (a supplement for the growth and expression of post-mitotic neurons and tumor cells of neuronal phenotype, Gibco) and 20 μg/ml basic Fibroblast Growth Factor (bFGF).

Cell Feeding and Passage

In order to maintain cells, the entire volume of defined media was replaced every 5 to 20 days as determined by the rate of nutrient depletion and/or waste buildup in the media as indicated by changes in media color. Cells were passaged (divided into fresh plates containing poly-L-omithine, as above) at 1:1 to 1:4 ratios using ATV solution once every 7 to 20 days depending on cell density (the ideal range is from 70% to 100% confluence). Cells are initially plated with Dulbecco's Modified Eagle Medium supplemented with 10% FBS up to 24 hours, media was then changed to above defined media contain bFGF.

Initially, these cells were grown in defined media and in the absence of growth factors known to promote propagation of Central Nervous System (CNS) stem cells. Subsequently, cells harvested from human fetuses were grown in the presence of mitogens such as bFGF, EGF or a combination of the two. These factors are known to cause proliferation of CNS stem cells. In order to classify the cell lines as stem cells, certain criteria, imposed by general guidelines as to what constitutes a stem cell, had to be met CNS stem cells should: express the nestin marker, perpetuate and retain their characteristics for as long as they are maintained in a suitable environment; and give rise to the different cells types of the nervous system.

Characterization Procedures

Nestin Expression

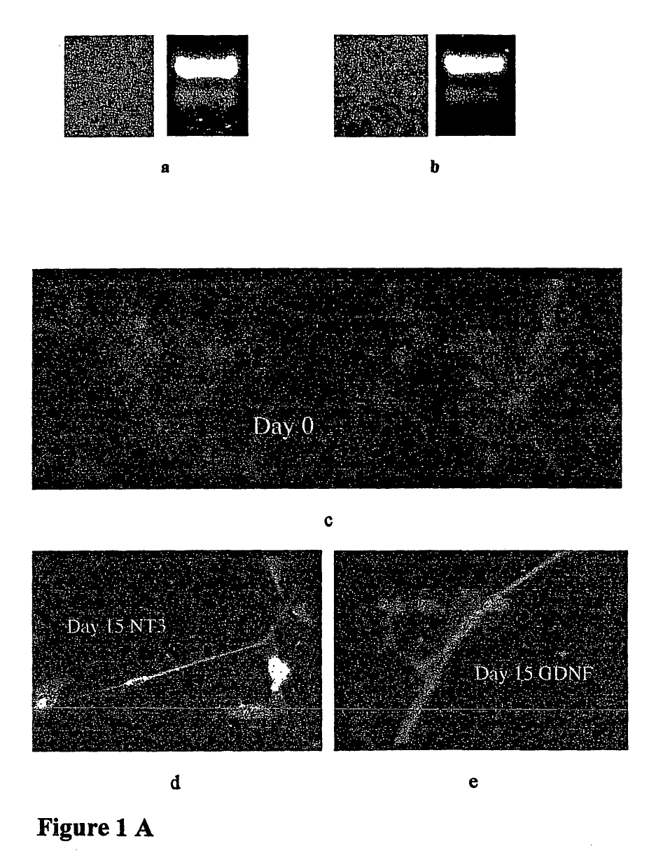

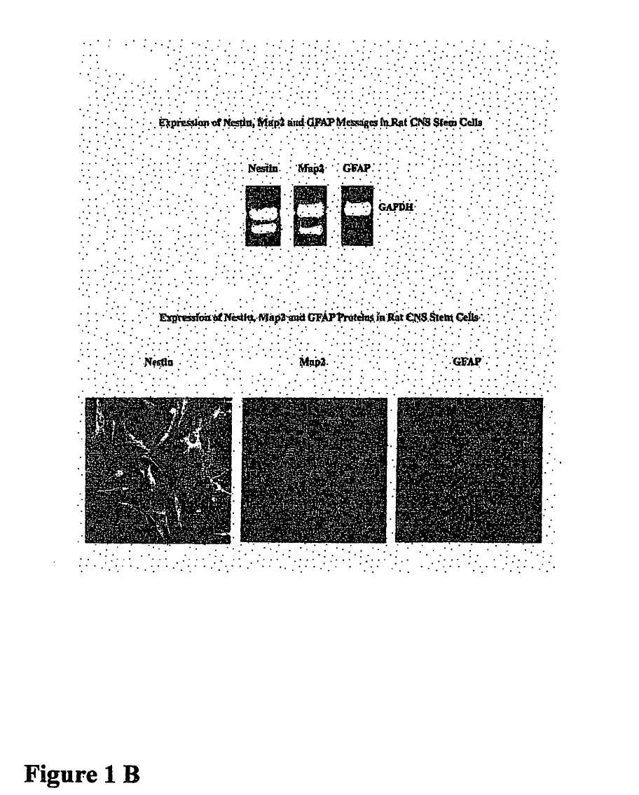

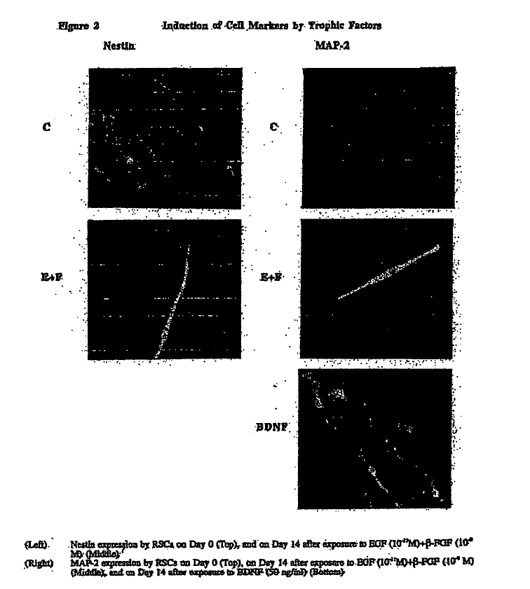

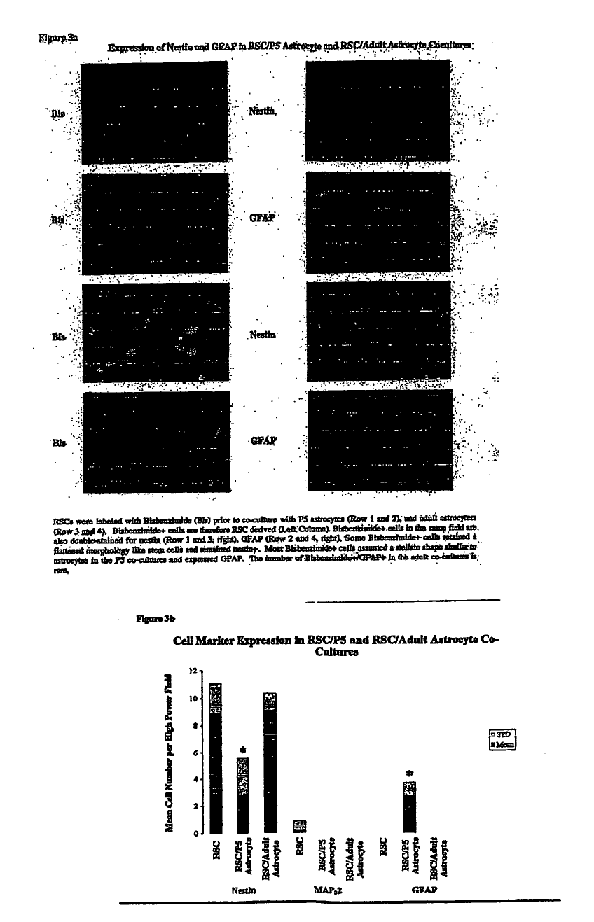

Essentially, there are two methods to detect the expression of certain genes within a cell or tissue. One method is to direct an antibody against the expressed protein, and the other is to search for the expressed gene itself Nucleotide primers, designed to amplify a part of the human nestin gene, were constructed to detect the presence of human nestin expressed by extracted RNA Almost all cell lines grown in the presence of basic Fibroblast Growth Factor (bFGF) and harvested in accordance to the protocol described hereinabove revealed that the nestin gene was actively expressed FIG. 1A(a) shows a field of stem cells on the left, and the RT-PCR bands for GAPDH (top) and Nestin (bottom) on the right FIG. 1A(b) shows a similar fields for a different strain of cells. The photos and RT-PCR data were obtained near the end of our study, and show that after 26 months these cells expressed nestin and were able to proliferate and retain a morphology characteristic of human CNS stem cells.

Perpetual Propagation in an Undifferentiated State

Propagation without differentiation of several cell lines was maintained for 26 months, approximately 54 passages, in culture. The lines retained nestin expression and the ability to perpetuate in a consistent manner. These lines were passaged once every two weeks and maintained their ability to grow and divide, for the duration of the experiment Regular CNS cells do not proliferate in culture.

Differentiation into CNS Cell Fates

Under the right conditions the stem cells gave rise to markers, both message and protein, such as the GFAP marker for Glia and Map2 for neurons. The procedures below show that these cells were induced by certain factors to differentiate to neuronal and glial type.

Induced Differentiation

CNS stem cells were exposed to NT3 and for a period of 15 days and stained for Map2, a marker characteristic of neuronal cells. FIG. 1c shows two control fields of cells stained with the Map2 antibody. FIG. 1d is of CNS stem cells treated with NT3. Both factors show an elevated amount of Map2 expression indicating differentiation towards a neuronal fate.

Human Fetal CNS Stem Cells and Markers

Co-culture experiments involving human CNS stem cells and cells from other germ layers from human or trans-species were conducted A rat model of these cell lines was established with dramatic results as described hereinbelow (rat CNS co-cultured with rat pituitary, pancreatic, glial and neuronal cells).

Rat Cell Markers

Cell marker expression was characterized by Reverse Transcriptase Polymerase Chain Reaction (RT-PCR) and Immunocytochemistry (IM). The same methods were used to characterize RSC differentiation into neural and extra-neural tissues.

Quantitative Reverse Transcriptase-Polymerase Chain Reaction

Characterization of Nestin, MAP-2, GFAP, Lhx-3, Pit-1, Prolactin, GATA-4 and Cardiac Myosin heavy chain expression using quantitative reverse transcriptase-polymerase chain reaction (RT-PCR) was done in RSC Cells.

RSCs were seeded in duplicate at approximately 1×10⁵ cells/60 mm tissue culture plate and evaluated for the expression of Nestin, MAP-2, GFAP, Lhx-3, Pit-1, Prolactin, GATA4 and Cardiac Myosin Heavy Chain at the message level. RNA was extracted from the cells using Trizol (Gibco BRL Life Technologies, Grand Island, N.Y.). Three μg of RNA were reverse-transcribed into cDNA using the Superscript II Preamplification System (Gibco BRL Life Technologies, Grand Island, N.Y.). Quanitative PCR, using glyceraldehyde-3-phosphate dehydrogenase (GAPDH) as an internal control, was conducted to assay the level of each message. The PCR (25 μl) included: 1× PCR buffer (Gibco BRL Life Technologies, Grand Island, N.Y.), 2 mM MgC₂ (Gibco), 0.4 mM dNTPs (Gibco), 0.2 μM oligo primers, 0.5 μl of the RT product, and 1.5 units Amplitaq (Perkin Elmer). The PCR was carried out as follows: 95° C. for 3 min, 35 cycles of reaction at 94° C. for 1 min; 54° C. for 1 min; 72° C. for 2 mm; and 72° C. for 10 min. The primers, selected for rat nestin, were MAP-2, GFAP, GAPDH (Gibco BRL Life Technologies, Grand Island, N.Y.), Lhx3, Pit-1, Prolactin, GATA-4 and Cardiac Myosin Heavy Chain:

Nestin sense primer
ACTGAGGATAAGGCAGAGTTGC
Nestin anti-sense primer.
AGTCTTGTTCACCTGCTTGG
Map-2 sense primer
AATTGCCTTCCTCATTCG C
Map-2 anti-sense primer
TGTCTTCCAGGTTGGTACCG
GFAP sense primer
ACCGGTGGAGATAACTTGG
GFAP anti-sense primer
ACCGGTGGAGATAACTTGG
GAPDH sense primer
TTCAACGGCACAGTCAAGG
GAPDH anti-sense primer
CATGGACTGTGGTCATGAGC
Lhx3 sense primer
AGAGCGCCTACAACACTTCG
Lhx3 anti-sense primer
CTTGTCGGACTTGGAACTGC
Pit-1 sense primer
AGACACTTTGGAGAGCACAGC
Pit-1 anti-sense primer
GGAAAGGCTACCACACATGG
Prolactin sense primer
GACTAGGTGGAATCCATGAAGC
Prolactin anti-sense primer
CTTCATCAACTCCTTGCAGG
GATA-4 sense primer
CAG CAG CAG TGA AGA GAT GC
GATA-4 anti-sense primer
GTT CCA AGA GTC CTG CTT GG
Alph-Cardiac Myosin HC sense primer
TCC ATT GAT GAC TCC GAG G
Alph-Cardiac Myosin HC anti-sense primer
TTG TCA GCA TCT TCT GTG CC

The RT-PCR products were analyzed in a 2% agarose gel after staining with ethidium bromide.

Immunocytochemical Characterization of Markers

RSC, RSC/Atrocyte, RSC/C6 Glioma, and RSC/GH₃ Co-Cultures

Cells on glass coverslips were fixed with 4% paraformaldehyde (in PBS) for one hour at 22° C., exposed to Triton X-100 (0.5% in PBS) for 10 minutes, and treated with blocking buffer (5% normal goat serum in PBS) for 30 minutes at 22° C. For characterization of the natural development of isolated RSCs as well as their development upon exposure to glial cells, cultures were reacted with one of the following primary antibodies: (1) a mouse monoclonal antibody against nestin at 1:500 dilution (Pharmingen, San Diego, Calif.), (2) a rabbit polyclonal antibody specific for cow GFAP at 1:200 dilution (Dako, Carpinteria, Calif.), or (3) a mouse monoclonal antibody specific for MAP-2 at 1:200 dilution (Pharmingen, San Diego, Calif.). Controls consisted of staining with PBS/5% NGS from which the primary antibodies were omitted as well as preimmune serum For characterization of the expression of pituitary factors and hormones, cultures were exposed to one of the following primary antibodies: (1) a mouse monoclonal antibody against nestin at 1:500 dilution (Pharmingen, San Diego, Calif.), (2) a goat anti-prolactin antibody at 1:200 dilution (Santa Cruz, Santa Cruz, Calif.), (3) a rabbit anti-human growth hormone antibody (Dako, Carpinteria, Calif.) at 1:400 dilution, or (4) a rabbit anti-Pit 1 antibody at 1:200 dilution (Santa Cruz, Santa Cruz, Calif.).

After one hour at 37° C., the cells were washed extensively with PBS. Cells were then reacted for 30 minutes at 37° C. with a second antibody which is either (1) a goat anti-rabbit IgG conjugated to fluorescein (1:100 dilution in PBS/5% NGS) (Sigma, St Louis, Mo.); or (2) a goat anti-mouse IgG conjugated to rhodamine (1:25 dilution) (Sigma, St Louis, Mo.), or (3) rhodamine conjugated goat anti-rabbit IgG (1:80 dilution), or (4) rhodamine conjugated rabbit anti-goat IgG (1:80).

In this analysis, RSCs were first identified by viewing the samples using a UV filter, which revealed the bisbenzimide labeled RSC nuclei as an intense light blue stained structure ( ). With the same view in place, the morphology and the expression of each specific factor were recorded for RSC derived cells. At least 5 to 10 random high power fields consisting of greater than 50 cells were examined under each condition for each cell marker. T-test comparisons between control and experimental groups were made. Significant differences (P<0.05) were indicated with an “*”.

Results

Initial primary brain cultures were composed of mostly small spindle cells mixed with cells of a fibroblastic and astrocytic morphology. With progressive culture, flat cells declined while the spindle cells predominated RSCs expressed the nestin message and protein consistent with their progenitor/stem cell identity (FIG. 1, Top and Bottom). Expression of the microtubule associated protein 2 (MAP-2) message was detected at a lower level while the number of MAP-2 immunostaining cells remained rare. The glial fibrillary acidic protein (GFAP) message was not seen and no cell stained for GFAP. Upon removal of bFGF from the culture medium, the number of nestin+cells declined while the number of GFAP+ and MAP-2+cells increased indicating progressive differentiation into the neurons and glia. For these reasons, these E12 RSCs are deemed to be stem cells.

Differentiation of RSCs into Central Nervous System Tissues

Induction of the Neuronal phenotype by Growth Factors

Method

RSCs in culture were exposed to a combination of growth factors for 14 days. At the end of the treatment period, cultures were fixed and analyzed for Map-2 expression using immunocytochemistry.

Results

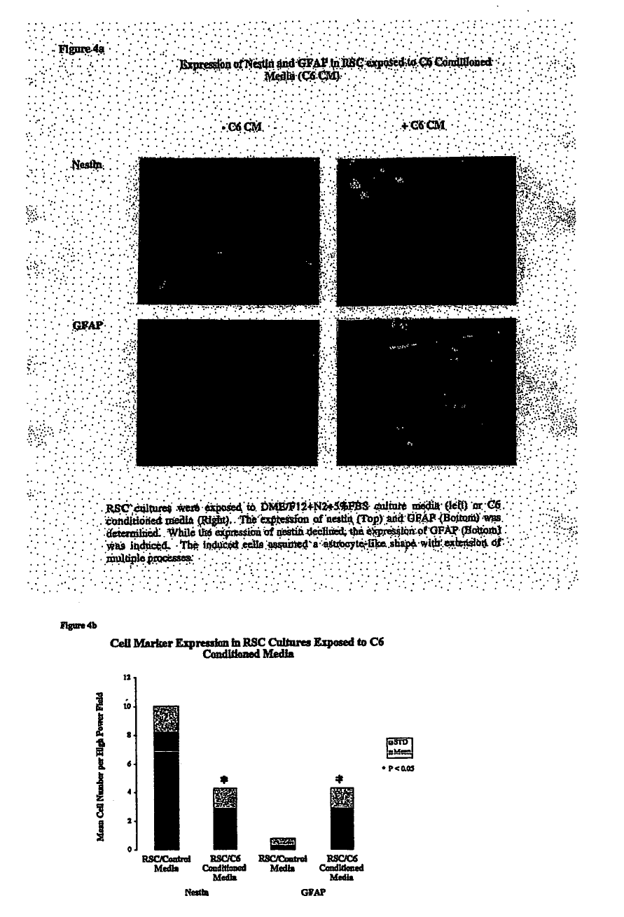

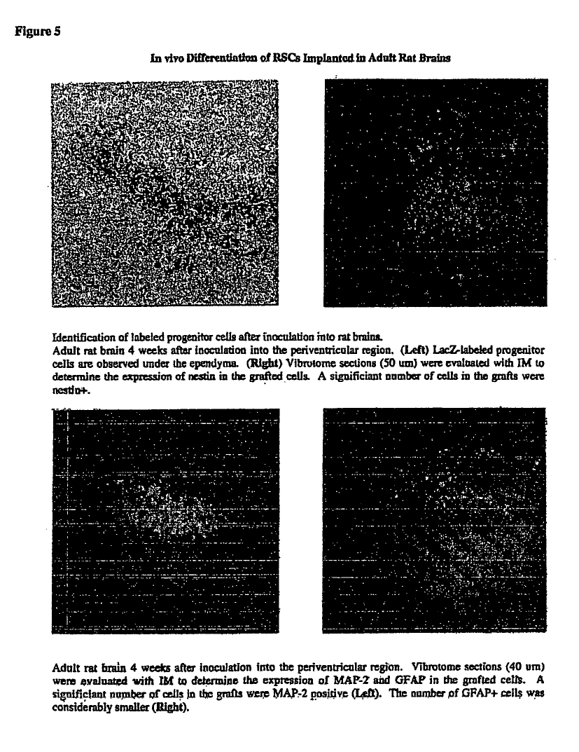

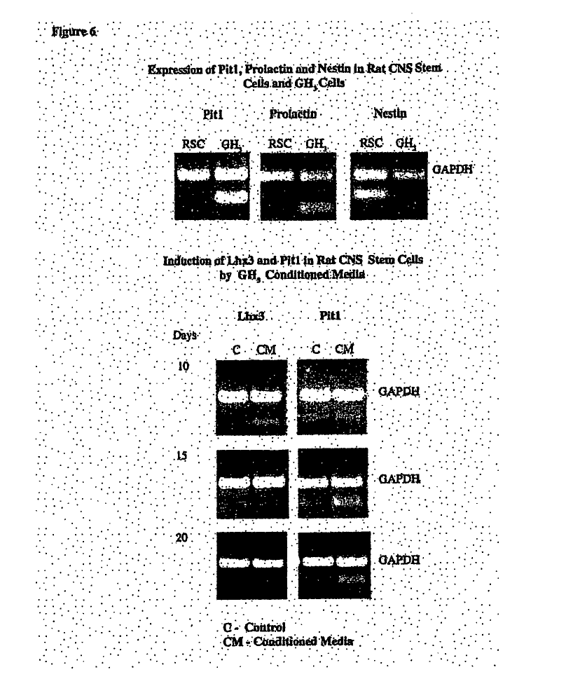

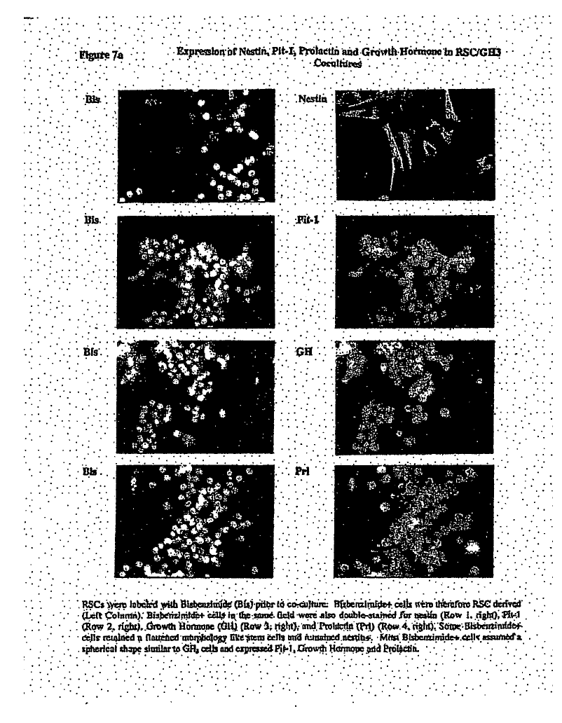

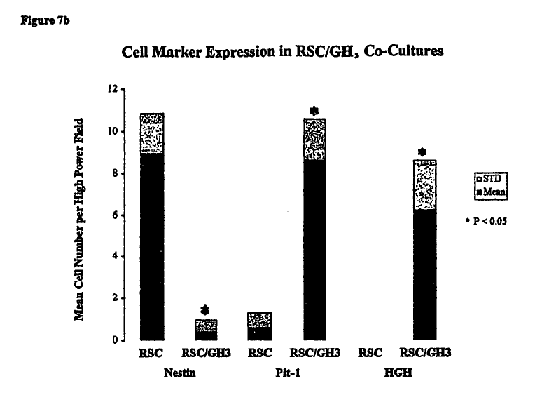

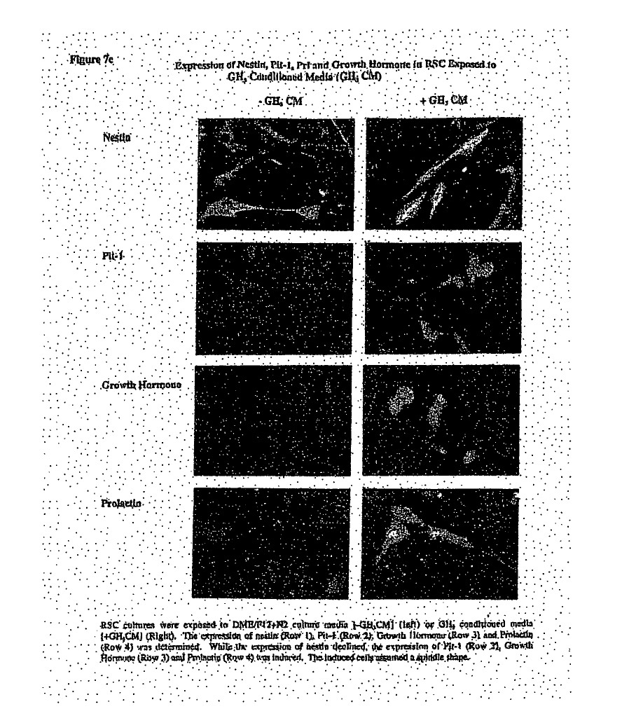

Nestin exhibited a bright cytoplasmic fibrillary pattern in most cells. GFAP and MAP-2 staining was not seen. MAP-2 expression was selectively induced by EGF (10⁻¹¹ M)+b-FGF (10⁻⁹ M) as well as BDNF (50 ng/ml) for 14 days suggesting induction of neuronal differentiation (FIG. 2**). Concurrently, nestin staining was reduced suggesting that defined factors can induce stem cells to develop selective cell types. RSCs exposed to developing and neoplastic glial cells were induced to manifest glial properties.

In, co-cultures, differentiating influences may be mediated by cell contact (e.g., connexons) or through the secretion of active substances. In order to distinguish these two mechanisms, RSCs were cultured for 21 days in media which had been exposed to C6 cells (C6 conditioned medium).

Induction of the Glial phenotype with co-culture with P5 neonatal astrocytes and C6 glioma cells as well as by exposure to C6 Conditioned Media

Methods

P5 neonatal astrocytes were generated from P5 neonatal rat brains and placed in tissue culture. In addition, C6 glioma cells were also placed in separate culture. Labeled RSCs were then co-cultured separately with each of these cell types.

Induction of the glial phenotype was also achieved by the exposure of RSCs to C6 cell conditioned media. Media exposed to C6 glioma (DME+10% FCS) cells were collected every six days and immediately filtered (0.2 um filter) without any further processing. Prior to use on the RSC cultures, the media was diluted 1:1 with DME/F12 medium supplemented with N2. To induce the RSCs, conditioned medium was added to each RSC culture maintained on PORN coated coverslips and dishes, and changed every three days. After 20 days of conditioning, RSC cultures were fixed for immunocytochemical analysis.

Results

On the co-cultures, RSCs slowly assumed both the morphology as well as the protein expression patterns (e.g., GFAP) of P5 astrocytes and C6 Glioma cells respectively.

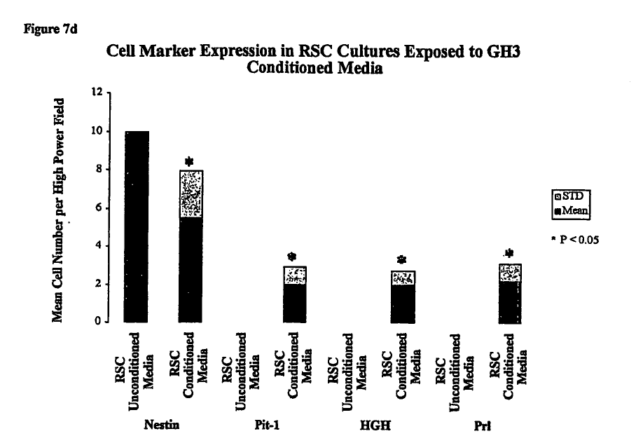

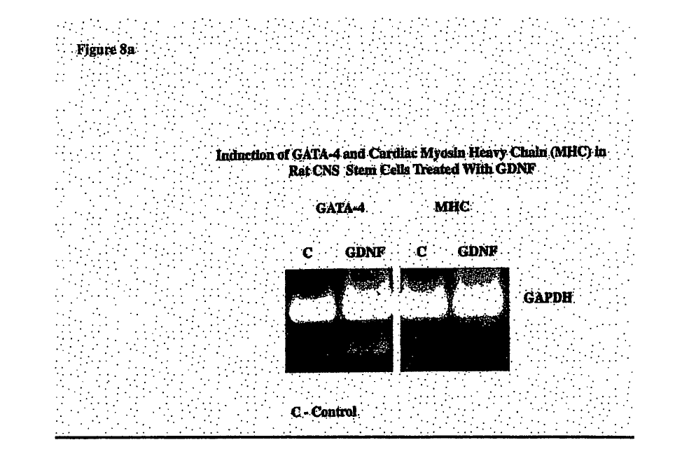

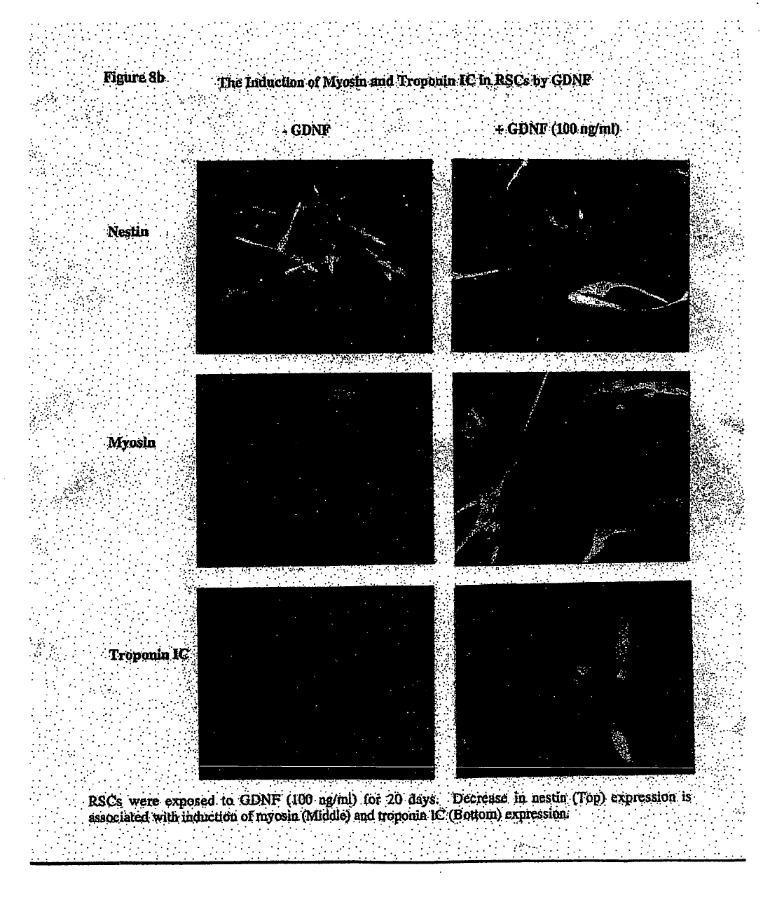

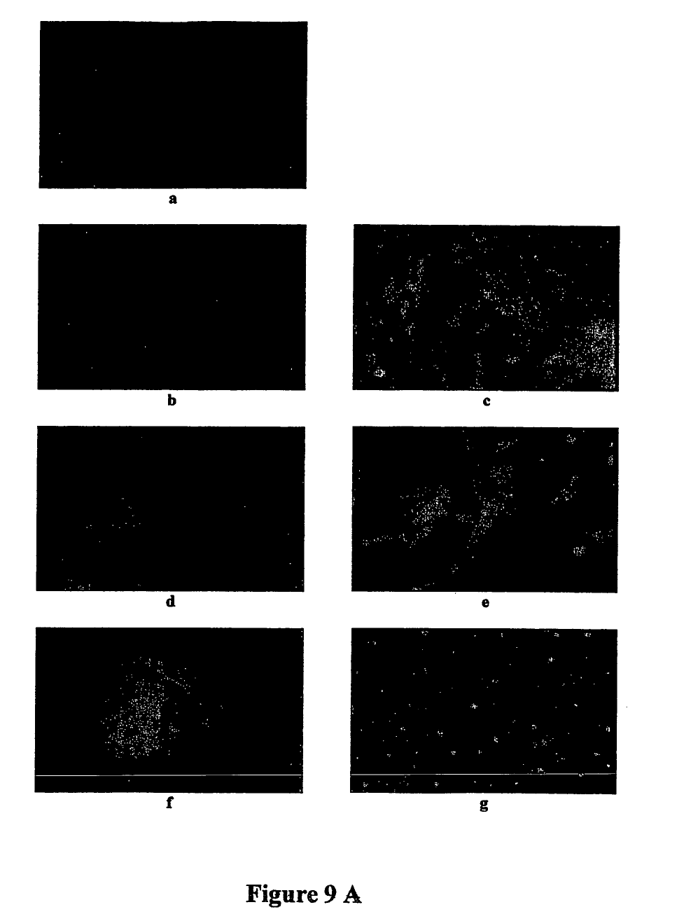

In the cultures exposed to C6 conditioned media, RSCs progressively assumed the stellate morphology characteristic of astrocytes (FIG. 4a) with the attendant increase in the number of GFAP+cells suggesting that factors in the conditioned media induced glial development (FIG. 4b). The expression of GFAP was confirmed by the induction of the GFAP message using RT-PCR.

Differentiation of RSCs into Central Nervous System Tissues after Plantation into the Rat Brain

Methods

Progenitor cells were first labeled by culture with a b-galactosidase expressing adenoviral vector. Infection at a MOI of 50 for 5 hrs lead to labeling of >80% of the cells. After three days, labeled cells were implanted stereotaxically into the periventricular region of adult rats or the frontal forebrains of P6 neonatal rats.

Results

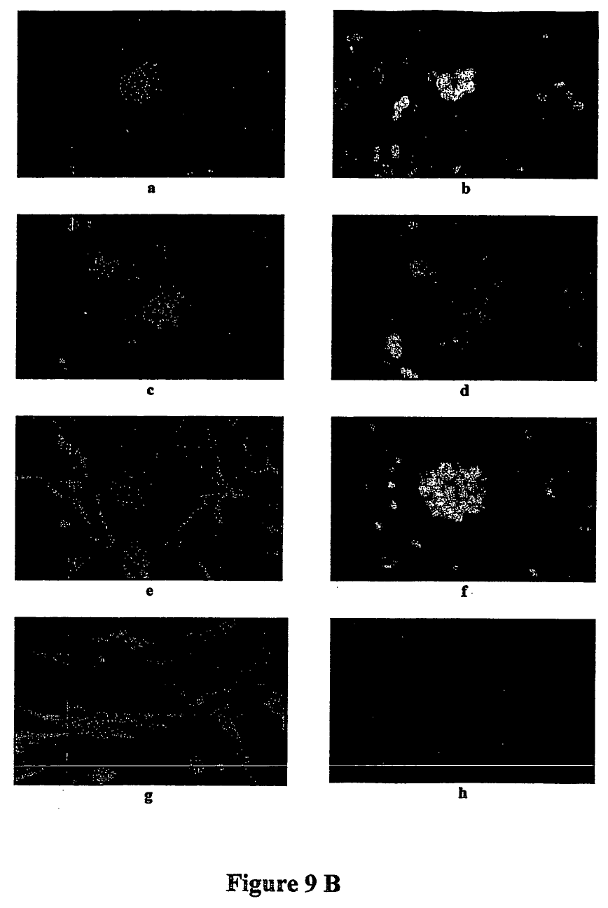

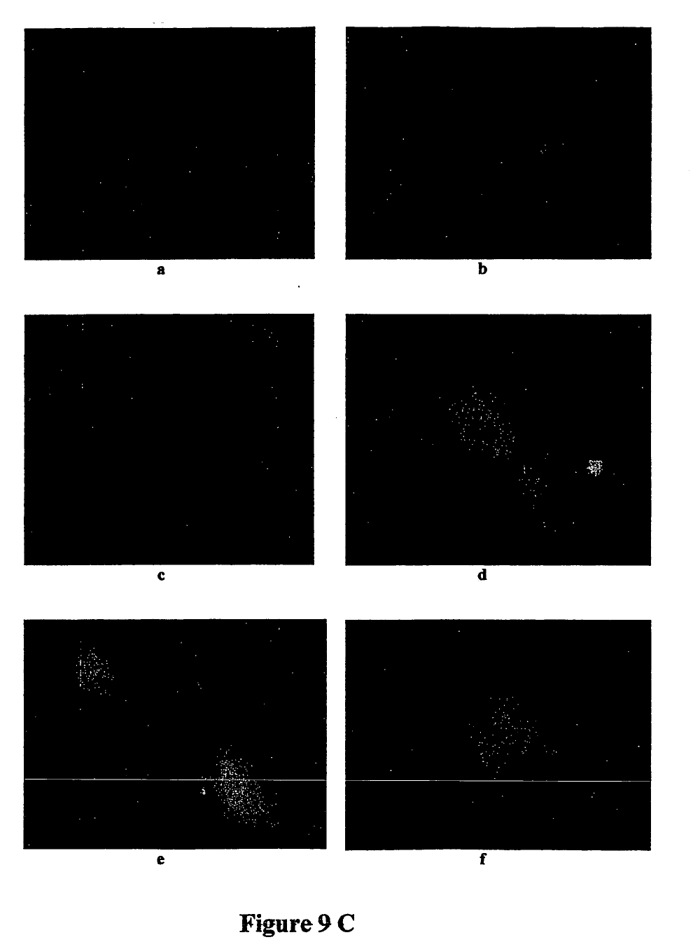

Brains showed LacZ+ cells at the injection sites and along the inoculation tract up to 4 weeks after implantation (FIG. 10***). IM analysis of vibrotome sections (40 μm) showed that a significant number of grafted cells were nestin+ and MAP-2+ (FIG. 5***). The number of GFAP+ cells was markedly smaller.

This demonstrated that fetal progenitor cells could survive for weeks after implant into adult and neonatal brains. All cells continued to express the introduced gene. While a large proportion of the cells remained nestin+, a significant number of cells had begun to express MAP-2 indicating development along the neuronal lineage. The number of cells that expressed GFAP was smaller, suggesting that the adult brain microenvironment was more supportive of neuronal than glialevelopment.

### EXAMPLE II

Differentiation of RSCs into Extra-Central Nervous System Tissues

The induction of glial development in RSCs by developing (P5) and transformed (C6) glial cells is consistent with the origin of RSCs in the CNS. Since CNS stem cells could also be induced to acquire fates outside the CNS (20, 21), it was further explored whether RSCs possess differentiation potentials beyond the ectoderm To this end, Bisbenzimide labeled RSCs were co-cultured for two weeks with GH₃ cells, an established rat pituitary tumor cell line (9).

Co-culture with MtT/W5 Rat Pituitary Tumor GH3 Cells

Methods

For co-culture, RSCs were first labeled for 3 days with 20 uM Bisbenzimide, (Sigma, St Louis, Mo.) which binds to DNA and fluoresces under an Ultraviolet filter. This allowed the identication of cells of RSC origin as Bisbenzimide+. On the day of co-culture, 10⁵ Bisbenzimide labeled RSCs were plated onto the GH₃ (ATCC, Rockville, Md.) cultures on PORN coated coverslips in DME+10% FCS. RSCs were first analyzed immediately after initial plating (Day 0) to provide a baseline characterization of cell marker expression. After two weeks, the samples were processed for IM analysis of the expression of nestin and pituitary related factors. As a negative control to evaluate pity specific hormone expression, RSCs not exposed to GH₃ cells were used. For positive control, GH₃ cells not co-cultured with RSCs were used. In these sets of co-cultures, two kinds of media were also used to control for the effects of the respective media: (1) DME/12 supplemented with N2 but not with any growth factor, (2) the Ham's/F12+15% HS+2.5% FCS medium used to maintain GH₃ cells.

Results

GH₃ cells demonstrated a spherical morphology and grew in culture as clumps of round cells, easily detachable from the growth surface. These cells expressed messages for the transcription factor Pit-1 and Prolactin (Pr1) but not nestin (FIG. 6, Top[FIG. 4 top M]). GH₃ cells were also immunoreactive with antibodies directed to Pr1, human growth hormone (hGH), and Pit-1. Therefore, the marker expression pattern and morphology of these cells were remarkably different from that of the RSCs described above. Upon plating of the RSCs onto the GH₃ cells, distinct populations representing the two cell types could be easily seen initially.

(FIG. 6) When RSCs were progressively co-cultured with GH₃, the number of spherical pituitary-like cells increased while that of stem cell morphology declined. By 21 days, the majority of cultured cells were indistinguishable from GH₃ cells. The presence of Bisbenzimide+nuclei identified these cells as cells of RSC origin (FIG. 7a). The occasional flat cells that showed blue nuclei and stained for nestin are identified as stem cells (FIG. 7a, Row 1). There were, however, some round cells in the cultures that were not positive for Bisbenzimide, suggesting they were GH₃ cells.

Co-cultures were stained for nestin, Pr1, hGH and Pit-1. Nestin+cells were invariably flat and positive for Bisbenzimide indicating that they were RSCs (FIG. 7a, Row 1). None stained for Pit-1, hGH or Pr1. On the other hand, round Bisbenzimide+ cells uniformly stained for Pit-1 (FIG. 7a, Row 2), hGH (FIG. 7a, Row 3) or Prl (FIG. 7a, Row 4) suggesting that they were derived from RSCs which have assumed the morphology, and Pr1, hGH and Pit-1 expression characteristic of GH₃ cells (FIG. 7b). In order to examine whether these trans-germ layer induction signals were also secreted as suggested by the glial induction studies, the effects on RSCs exposed to GH₃ conditioned medium were determined.

(FIG. 7ab)

Induction of the Pituitary Phenotype with G₃ Conditioned Media Methods

Media exposed to GH₃ (Ham's/F12+15% HS+2.5% FCS) cells were collected every six days and immediately filtered (0.2 um filter) without any further processing Prior to use on the RSC cultures, the media was diluted 1:1 with DME/F12 medium supplemented with N2. To induce the RSCs, conditioned medium was added to each RSC culture maintained on PORN coated coverslips and dishes, and changed every three days. After 20 days of conditioning, RSC cultures were fixed for immunocytochemical analysis.

Results

Upon exposure to GH₃ conditioned medium, RSCs did not show any morphologic change within the fist two weeks. During this period, expression of the messages for transcription factors, Lhx 3 and Pit-1, essential to pituitary development, was evaluated (FIG. 6 [4], Bottom). Lhx 3 was expressed by Day 10, while Pit-1 expression was not stimulated. By Day 15, no expression of Lhx 3 was observed while the expression of Pit-I was stimulated Expression of the Pit-I message was maintained up to Day 25 when RSC began to assume a more spindle morphology and the expression of pituitary hormones emerged. By the third week, selective cells began to assume a spherical shape and formed random clusters in a manner akin to GH₃ cells. After 20 days of conditioning, RSC cultures were fixed for immunocytochemical analysis. Cells which retained their flat morphology remained nestin positive (FIG. 5B, Row 1).

These spherical cells were negative for the nestin protein but expressed Pit-1 (FIG. 7c, Row 2), hGH (FIG. 7c, Row 3) or Prl (FIG. 7c, Row 4) (FIG. 7d). Therefore, RSCs exposed to GH₃ conditioned medium, like RSCs in GH₃ co-cultures, also acquired the morphologic and protein expression profiles characteristic of GH₃ cells. Cells which retained their flat morphology remained nestin positive (FIG. 7c, Row 1) and did not stain for Pit-1, Prl or hGH suggesting nonresponsiveness to the conditioned medium Thus, one means through which GH₃ cells exert their trans-differentiation effects is by the release of soluble factors. This observation was therefore identical to that in RSCs exposed to neonatal and transformed astrocytes. In both situations, RSCs were induced to transdifferentiate in a cell type specific manner by influences specified by cells derived from two separate germ layers.

(FIG. 7cd)

### EXAMPLE III

Differentiation into Pulsatile Cardiac Myocytes

Methods

In these investigations, RSCs in culture were exposed to DME/F12 medium supplemented with N2 and either 15% horse serum (HS) or GDNF at 50 or 100 ug/ml. At the end of the treatment period, the expression of cardiac cell specific transcriptional factors and markers were determined using RT-PCR and immunocytochemistry (IM). RT-PCR was performed as for characterization of other transcriptional factors and cell markers. For IM, RSC cultures exposed to horse serum and GDNF were fixed using the following primary antibodies: (1) a mouse monoclonal antibody against nestin at 1:500 dilution (Pharmingen, San Diego, Calif.), (2) a goat anti-troponin IC antibody at 1:100 dilution (Santa Cruz, Santa Cruz, Calif.), and (3) a rabbit anti-myosin antibody (Sigma, St Louis, Mich.) at 1:100 dilution.

Results

Upon exposure to GDNF alone or GDNF supplemented with Horse Serum, RSCs did not show any change in morphology within the first two weeks. By the third week, cells began to assume a more spindle morphology with many cells grouped together to form bundles which may be connected with long processes. Rhythmic contractile activities were observed in some bundles and were transmitted to the surrounding connected bundles as well. Cells from different bundles exhibited different contractile rates. Those exposed to GDNF at 100 μg/ml exhibited contractile activity sooner than those exposed to GDNF at 50 μg/ml.

After 5 days of conditioning, cultures were evaluated for the expression of GATA4, a transcriptional factor characteristic of cardiac development During this early period of conditioning, not only was GATA4 induction evident, a slight induction of cardiac myosin heavy chain was also seen (FIG. 8a). After 20 days, RSC cultures were fixed for IM analysis of the expression of troponin IC and myosin, markers of cardiomyocytes. Contractile cells were nestin- and showed significant reaction to antibodies directed to troponin IC and myosin (FIG. 8b). In these cultures, cells which retained their flat morphology remained nestin+ (FIGS. 8b, Top) and were uniformly not immunoreactive to antibodies specific for cardiac muscle antigens. Their number declined progressively. Fetal CNS stem cells were therefore similar to embryonic stem cells in being capable of generating contractile spindle-like cells that expressed troponin characteristic of cardiomyocytes when cultud in HS. Here, we demonstrated that a unique trophic factor, GDNF, acing alone can induce fetal CNS stem cells to transdifferentiate into a cell type derived from another germ layer, the mesoderm.

(FIG. 8ab)

### EXAMPLE IV

Differentiation into Pancreatic Tissues

Pancreatic Phenotype in RSCs was induced by Syrian Hamster pancreatic islet of Langehans beta cells (HT-T15).

Co-Culture with T15 Syrian Hamster Pancreatic Islet Cells

CNS stem cells give rise to glia and neurons in response to trophic factors, as described hereinabove. Their development in the brain also appears to be influenced by local micro environmental factors since both fetal and adult progenitor cells develop neuronal and glial phenotypes upon implantation into the fetal, newborn and adult brain Region specific development is observed when CNS stem cells are implanted into neurogenic areas of the adult brain such as the hippocampus where stem cells are found. This underlines the importance of a permissive environment which may provide modulating and/or instructive signals in the promotion of region specific development The identification of these permissive influences would be important in understanding the control of cell fate. In order to characterize these lineage-promoting influences, Inventors studied the developmental fate of rat fetal CNS stem cells (RSCs) exposed to the influence of cells with well-defined phenotypes such as Syrian Hamster pancreatic islet of Langehans beta cells (HT-T15). Here, Inventors show that RSCs co-cultured with HIT-T15 cells developed the morphologic and protein expression features characteristic of pancreatic cells. Therefore, RSCs possess differentiation potentials beyond their organ of origin and can be influenced to develop organ specific phenotypes through cell interaction.

Fetal Rat Central Nervous System Stem Cells

Clones of rat fetal CNS stem cells were established from the brains of E12 Fisher 344 rats (Harlan Sprague Dawley, Indianapolis, Ind.). The harvested ties were initially digested in trypsin/EDTA (Gibco BRL Life Technologies, Grand Island, N.Y.), dissociated by trituration, filtered through a sterile 60-mesh Nytex membrane, and plated onto poly-L-ornithine (PORN) (Sigma, St Louis, Mo.) coated culture dishes in Dulbecco's modified Eagle (DME) supplemented with 10% fetal calf serum (FCS) (DME+10% FCS) medium (Gibco BRL Life Technologies, Grand Island, N.Y.). After culture in serum supplemented medium for one day to facile cell adhesion to the culture dishes, the culture medium was changed to DME/F12 supplemented with N2 (insulin 500 ug/ml, transferrin 10,000 ug/ml, progesterone 0.63 ug/ml, putrascine 1611 ug/ml, and selenite 0.52 ug/ml)(Gibco BRL Life Technologies, Grand Island, N.Y.) and basic fibroblast growth factor (bFGF 1×10−9 M) (Sigma, St. Louis, Mo.). Cultures were maintained for more than twelve months and were passaged upon reaching confluence. Cells were identified as being stem cells by (1) continual expression of the stem cell marker, nestin, as shown by immunostaining with a mouse anti-rat nestin antibody (Pharmingen, San Diego, Calif.), (2) the ability for self renewal, and (3) the ability to generate neurons and glial cells upon withdrawal of bFGF and the introduction of specific trophic factors.

Fetal brain cell cultures were initially composed of a large number of small spindle cells mixed with cells of a fibroblastic and astrocytic morphology, characterized by large flat cells with an abundant cytoplasm. With progressive passage in culture, the number of flat cells declined while the spindle cells predominated. The self-renewing RSCs expressed the nestin message primarily consistent with their progenitor/stem cell property. Expression of the microtubule associated protein 2 (MAP-2) message was also detected but at a lower level. Glial fibrillary acidic protein (GFAP) messages were not seem Immunocytochemical staining of these cells confirmed the message expression patterns and showed RSCs to be nestin positive. The number of MAP-2 positive cells remained rare. GFAP+cells were not detected. Upon removal of bFGF from the culture medium, the number of nestin+cells declined while the number of GFAP+ and MAP-2+ cells increased indicating progressive differentiation of the progenitor cells into the neuronal and glial phenotypes. For these reasons, the cells isolated from the E12 fetal brains were deemed consistent with stem cells.

Co-Culture

In order to define the effects of the environment on the differentiation potential of RSCs, RSCs were co-cultured with HIT-T15 cells. T15 cells are an established Syrian Hamster pancreatic islet of Langerhans beta cell line (ATCC, Rockville, Md.) which were maintained in Ham's F12K medium supplemented with 10% horse serum (HS) and 2.5% fetal calf serum (FCS). Three days prior to co-culture with HIT-T15 cells, RSCs were labeled with 20 μM Bisbenzimide (Hoechst 33258, Sigma, St Louis, Mo.) in order to label their nuclei. Bisbenzimide binds specifically to the adenine-thymidine regions of DNA and fluoresces under an Ultraviolet filter. For co-culture, HIT-T15 cells were initially plated onto PORN coated cover slips or culture dishes at a density of about 1×106 cells per dish One day later (the day of co-culturing), the Bisbenzimide labeled RSCs were harvested and plated onto the HIT-T15 cultures at a density of about 1×10⁵ cells per dish Fresh media was supplied once a week. After three weeks in co-culture, the samples were fixed for immunocytochemical analysis. As negative controls, to evaluate the expression of insulin, RSCs analyzed immediately after initial plating (Day 0) and RSCs maintained in Hams/F12+10% HS+2.5% FCS medium in the absence of HIT-T15 Cells for the duration of the experiment were used. For positive control HIT-T15 cells not co-cultured with RSCs were used.

T15 cells grew in culture as islands of granular cells. HIT-T15 cells were immunoreactive with antibodies directed to rat insulin Therefore, the marker expression and morphology of these cells were remarkably different from that of the rat CNS stem cells as described above. Upon plating of the RSCs onto the HIT-T15 cells, initially, distinct populations representing the two cell types could be easily seen With progressive culture the RSCs in between the HIT islands became elongated and dense, same as in the RSC control plates, revealing the effects of the serum-supplemented media. As for the RSCs in proximity to the HITs, they were of less obvious morphology, RSCs growing within or on HIT islands were discernable in the beginning but later blended into the overall morphology of the islet cluster.

Cultures were stained with antibodies specific for nestin and insulin The granular islands stained uniformly for insulin and not for nestin A majority of these clusters revealed nuclei that were positive for bisbenzimide, suggesting a RSC origination In addition, we found certain cells that were positive for insulin but lacked the Bisbenzimide nuclear stain, suggesting a HIT-T15 origination. None of the cells outside the clusters expressed insulin, yet, a majority of them stained for nestin and all had Bisbenzimide positive nuclei.

As a control, RSCs grown in serum supplemented media in the absence of T15 cells were stained for both insulin and nestin. A majority of them stained positive for nestin but non stained positive for insulin.

Therefore, RSCs have not only changed their morphology upon co-culture with HIT-T15 cells, they have also assumed the insulin expression profile characteristic of Hr-T15 cells. One mechanism would be the transmission of trans-differentiation signals from the HIT-T15 cells to RSCs through direct cellular contact Alternatively, HIT-T15 could secrete transforming substances into the medium which were active on the RSCs, inducing them to develop phenotypes (both morphology and protein expression patterns) characteristic of HIT-T15 cells.

Induction with HIT Conditioned Media

It was also shown that RSCs exposed to media conditioned with HIT-T 15 cells developed the morphologic and protein expression features characteristic of pancreatic cells. Therefore, RSCs possess differentiation potentials beyond their organ of origin and can be influenced to develop organ specific phenotypes through the action of soluble factors secreted by other cells.

Immunocytochemical staining of RSC cells confirmed the message expression patterns and showed RSCs to be nestin positive (FIG. P1). Induction of the Pancreatic Phenotype in RSCs by media conditioned with Syrian Hamster pancreatic islet of Langehans beta cells (HIT-T15) In order to define the effects of the environment on the differentiation potential of RSCs, RSCs were exposed to media conditioned with HIT-T15 cells. HIT-T15 cells are an established Syrian Hamster pancreatic islet of Langerhans beta cell line (ATCC, Rockville, Md.) which were maintained in Ham's F12K medium supplemented with 10% horse serum (HS) and 2.5% fetal calf serum (FCS).

T15 cells grew in culture as islands of granular cells. T15 cells were immunoreactive with antibodies directed to Rat Insulin. Therefore, the marker expression and morphology of these cells were remarkably different from that of the rat CNS stem cells as described above. (FIG. P2, P3).

Medium exposed to HIT-T15 cells was collected every three days and immediately filtered (0.2 um filter). To induce the RSCs, HIT-T15 conditioned medium was added to each RSC culture maintained on PORN coated coverslips and dishes The conditioned media was changed every three days. Cells were examined daily for morphologic changes using an inverted Nikon microscope. After 21 days of conditioning, RSC cultures were fixed for immunocytochemical analysis. As a negative control, RSCs were exposed to identical medium that was not conditioned by HIT-T15 Cells.

Upon exposure to HIT-T15 conditioned medium, RSCs did not show any change in morphology in the first two weeks. By the third week, selective cells began to assume a granular shape and formed clusters in a manner akin to HIT-T15 cells. Other cells also began to acquire more spindle morphology FIG. P3 as reference).

Cells grown in media not conditioned by HIT-T15 maintained a more flat morphology, no granular or spindle shapes cells were produced and no insulin positive cells were detected (FIG. P4, P5, P6).

Cells grown in Media conditioned by HIT-T15 gradually acquired the spindle and granular morphology and were insulin positive. (FIG. P7, P8, P9) Only cells with flat morphology remained Insulin negative (FIG. P9) This suggests that they have not responded to the effects of the conditioned medium. Therefore, RSCs exposed to HIT-T15 conditioned medium demonstrated the morphologic and protein expression profiles characteristic of HIT-T15 cells. This suggests that HIT-T15 cells exert their tans-differentiation effects by the release of soluble factors. These factors, known or unknown, are of great importance.

(FIGS. P1-P9)

Discussion

These results demonstrated that CNS stem cells could be influenced in co-cultures to acquire phenotypes characteristic of one of the CNS constituents, the astrocytes. Both P5 astrots and C6 glioma cells exerted influences randomly throughout the co-cultures. This finding together with the failure of adult astrocyte cultures to behave similarly suggest that these transdifferentiation influences acted through instructive mechanisms instead of permissive mechanisms. The induction of astrocytic properties in RSCs by media which had been conditioned by C6 cells demonstrates that the factor(s) responsible for this transdifferentiation may be secreted by the C6 cells. Since C6 glioma cells grow aggressively, it is likely that these cells would generate the greatest influence on their environment perhaps through paracrine processes. The observations described herein are consistent with this and further support the hypothesis that these effects were instructive rather than permissive in nature.

In order to determine whether CNS stem cells could only be induced to differentiate into cells endogenous to the CNS, experiments were conducted in which RSCs were exposed to another well defined cell type, GH₃, which was derived from a different germ layer. RSCs exposed to GH₃ cells in co-cultures as well as to GH₃ conditioned media acquired the same morphology and protein expression profile as GH₃ cells. Furthermore, in RSCs exposed to GH₃ conditioned media, the transcription factors, Lhx 3 and Pit-1, essential to pituitary development, were activated in a temporal specific manner (prior to the expression of pituitary hormones), i.e., that these factor(s) were activating a pituitary specific differentiation pathway.

In addition to GH3 cells, we also co-cultured RSCs with Syrian Hamster pancreatic Islet HIT-15 cells. In a manner similar to co-cultures with P5 astrocytes, C6 glioma cells and GH3 cells, RSCs assumed the morphology and insulin expression pattern as HIT-15 cells. When RSCs were exposed to HIT-15 conditioned media, the expression of the insulin message appeared to be preceded by the expression of Isl-1, a transcriptional factor associated with pancreatic development.

In the glial and GH₃ studies, RSCs were exposed to environments composed of cellular and ill-defined secreted influences. Of these factors, GDNF alone induced RSCs to express biologic (contractile) and protein expression properties characteristic of cardiomyocytes, a cell type derived from yet another germ layer, the mesoderm. In this case, one factor appears adequate to activate this effect. In cardiac myocytes, it is likely that GDNF activates the transcription of a member of the GATA gene family, and particularly GATA4, a transcription factor essential to the development of the cardiac phenotype (26-28). Upon activation, GATA4 binds to the promoter/enhancer regions of cardiac specific genes such as cardiac-specific brain natriuretic protein (BNP), cardiac troponin C (cTpC) (26), and □□-myosin heavy-chain (□□□ C)(30). Cardiac development star early in embryogenesis with the initial commitment of anterior lateral plate mesodermal cells to the cardiac lineage. These committed precursor cells then differentiate into cardiac myocytes. This precedes the morphogenetic process of heart formation. The findings described herein support the determination that cardiac differentiation may initiate with locally active factors such as GDNF which activates transcription factors such as GATA-4 in pluripotential stem cells leading to their commitment to the cardiac lineage through expression of cardiac specific proteins.

Taken together, these observations indicate that even though the stem cells used in the experiments were all derived from the CNS and thus appeared to be committed to the development of the CNS, they do not seem to be restricted to a defined developmental fate. Each cell is supposed to contain all the genetic components characteristic of a specific organism and therefore is potentially capable of generating every organ in that organism should the requisite set of genes be activated. These observations therefore indicate that partially committed stem cells, for example, CNS stem cells may retain pluripotentiality and can be redirected to develop into other cell types not found in the brain provided the correct set of stimuli is present In this sense, the differentiation potential of stem cells may emend beyond the developmental divisions separating organs thereby illustrating that the developmental potential of stem cells is more universal than previously thought.

In accordance with these and other possible variations and adaptations of the present invention, the scope of the invention should be determined in accordance with the following claims, only, and not solely in accordance with that embodiment within which the invention has been taught.

### REFERENCES

(1) R McKay, Science 276,66 (1997).
(2) A. L. Vescovi, B. A Reynolds, D. D. Fraser, E. Weiss, Neuron 11, 951 (1993).
(3) F. H. Gage, J. Ray, L. J. Fisher, Ann Rev Neurosci 18,159 (1995).
(4) K Campbell, M. Olsson, A Bjorklund, Neuron 15,1259 (1995).
(5) E. Y. Snyder, D. L. Deitcher, C. Walsh, S. Arnold-Aldea, E. A Hartwieg, E. A., C. L. Cepko, Cell 68,33 (1992).
(6) F. H. Gage, P. W. Coates, T. D. Palmer, H. G. Kuhn, L. J. Fisher et al, Proc Natl Acad Sci 92,11879 (1995).
(7) C. N. Svendsen, D. J. Clarke. A. E. Rosser, S. B. Dunnett, Exp Neurology 137,376 (1996).
(8) R. A. Fricker, M. K. Carpenter, C. Winkler, C. Greco, M. A Gates, A Bjorklund, J Neurosci 19,5990 (1999).
(9) A. H. Tashjian, Jr, Y. Yasumura, L. Levine, G. H. Sato, M. L Parker, M L, Endocrinology 82,342 (1968).
(10) H. A. Ingraham, R. P. Chen, H. J. Mangalam, H. P. Elsholtz, S. E. Flynn, et al. Cell 55(3), 519(1988).
(11) X. He, M. N. Treacy, D. M. Simmons, H. A Ingraham, L. Swanson, M. G. Rosenfeld, Nature 340, 35 (1989).
(12) D. M. Simmons, J. M. Voss, H. A Ingraham, I. M. Holloway, R. S. Broide, M. G. Rosenfled, Genes Dev. 4,695 (1990).
(13) M. Treier, M. G.-Rosenfeld, Current Opiniion Cell Biology 8,833 (1996).
(14) S. J. Rhodes, G. E. DiMattia, M. G. Rosenfeld, Current Opinion in Genetics and Development 4,709 (1994).
(15) H. Z. Sheng, A. B. Zhadanov, B. Mosinger, Jr., T. Fuji S. Bertuzzi, S. et al, Science 272,1004 (1996).

(16) M. W. Sornson, W. Wu, J. S. Dasen, S. E. Flynn, D. J. Norman et al, Nature 384,327 (1996).
(17) S. Li, E. B. Crenshaw III, E. J. Rawson, D. M. Simmons, L. W. Swanson et al, Nature 347,528 (1990).
(18) T. D. Palmer, J. Takahashi, F. H. Gage, Mol Cell Neurosci 8,389 (1997).
(19) K. McCarthy, J. deVellis, J Cell Biology 85,890(1980).
(20) C. E. Henderson, H, S, Phillips, R. A Pollock, A. M. Davies, C. Lemeulle et al, Science 266,1062 (1994).
(21) M: Trupp, N. Belluardo, H. Funakoshi C. F. Ibanez, J Neurosci 17,3554 (1997).

(22) J. P. Golden, J. A. DeMaro, P. A. Osborne, J. Milbrandt, J., E. M. Johnson, Jr., Exp Neurol 158,504 (1999).

(23) Y. Kubo, J Physiol 442,743 (1991).
(24) C. R Bjornson, R. L. Rietze, B. A Reynolds, M. C. Mafli, A. L. Vescovi Science 283,534 (1999).
(25) D. L. Clarke, C. B. Johansson, J. Wilbertz, B. Veress, E. Nilsson, H. Karlstrom, U. Lendahl, J. Frisen, Science 288,1660 (2000).
(26) J. Ericson, S. Norlin, T. M. Jessell, T. Edlund, Development 125,1005 (1998).
(27) H. S. Ip, D. B. Wilson, M. Heikinhemio, Z. Tang, C—N. Ting, M. C. Simon, J. M. Leiden, M. S. Parmacek, Mol Cell Biol 14,7517 (1994).
(28) A. C. Layerriere, C. MacNeill, C. Mueller, R. E. Poelmann, J. B. E. Burch, T. Evans, J Biol Chem 269,23177 (1994).
(29) C. Grepin, L. Robitaille, T. Antakly, M. Nemer, Mol Cell Biol 15,4095 (1995).
(30) C. Grepin, L. Dagnino, L Robiille, L. Haberstroh, T. Antakly, M. Nemer, Mol Cell Biol 14,3115 (1994).

1. A pluripotent mammalian central nervous system (CNS) stem cell line, comprising:
stem cells isolated from fetal, neonatal or adult brain having the capacity of proliferating perpetually in an undifferentiated state as CNS stem cells and differentiating into functional cells of the ectoderm, mesoderm or endoderm tissue groups, wherein said capacity is manifest when said stem cells are grown in an environment selected from the group consisting of an environment comprising cells selected from one of said tissue groups, an environment comprising one or more stimulating factors produced by selected cells from one of said tissue groups, an environment comprising one or more stimulating factors from a non-cell source, and
an environment comprising the absence of one or more stimulating factors.

2. A cell line according to claim 1, wherein the presence or absence of stimulating factors or signals from other mammalian cell types induces said stem cells to differentiate into neurons and glia.

3. A cell line according to claim 2 wherein the absence of beta Fibroblast Growth Factor in the growth medium induces said stem cells to differentiate into cells with glial properties.

4. A cell line according to claim 1, wherein stimulating factors or signals from adjacent endocrine cell types induces said stem cells to differentiate into endocrine cells.

5. A cell line according to claim 4, wherein the induced endocrine cells produce insul.

6. A cell line according to claim 4, wherein the differentiated cells are insulin-producing pancreatic beta cells.

7. A cell line according to claim 1, wherein said stem cells differentiate into endocrine cell types having the capability to produce one or more members of the group of pituitary factors consisting of growth hormone, prolactin, and pit1.

8. A cell line according to claim 7, wherein said differentiation is induced by factors or signals isolated from mammalian pituitary cells.

9. A cell line according to claim 7, wherein the differentiation is induced by contact with mammalian pituitary cells.

10. A cell line according to claim 7, wherein the endocrine cells are pituitary cells.

11. A cell line according to claim 1, wherein the stem cells differentiate into cardiac cell types through the exposure of said stem cells to horse serum and GDNF.

12. A cell line according to claim 11, wherein the cardiac cell types are pulsatile cardiac cells.

13. A cell line according to claim 12, wherein the pulsatile cardiac cells express one or more cardiac transcription factors.

14. A cell line according to claim 13, wherein the transcription factor is a member of the group consisting essentially of GATA4, myosin, or troponin IC.

15. A cell line according to claim 1, wherein said stem cells differentiate into glial cell types in the presence of other mammalian cell types.

16. A cell line according to claim 15, wherein said stem cells differentiate into glial cell types in the presence of mammalian Post Natal-5 days primary astrocytes culture.

17. A cell line according to claim 15, wherein said stem cells differentiate into glial cell types in the presence of mammalian glioma cultures.

18. A cell line according to claim 1, wherein said stem cells differentiate into glial cell types in the presence of isolated factors and or signals from other mammalian cell types.

19. A cell line according to claim 1, wherein said stem cells are capable of differentiating into neurons in the presence or absence of factors or signals from other mammalian cell types.

20. A cell line according to claim 20, wherein said stem cells respond to the presence of EGF and bFGF by differentiating into neurons expressing microtubule associated protein 2 (Map-2) marker.

21. A cell line according to claim 20, wherein the cells respond to the presence of BDNF by differentiating into neurons expressing Map-2 marker.

22. A method for inducing trans-differentiation of pluripotent stem cells into other cell types, comprising:
harvesting the pluripotent stem cells from tissues and/or organs;
placing the harvested cells into cell culture;
culturing the cells under conditions suitable for maintaining pluripotency;
contacting the cultured pluripotent cells with differentiation-inducing factors; and
determining differentiation into a particular cell type.

23. The method according to claim 22, wherein the harvesting comprises teasing or trituration of fetal, neonatal or adult CNS tissue.

24. The method according to claim 22, wherein said harvested cells are placed on poly-L-omithine coated culture plates.

25. The method according to claim 22, wherein the contacting is accomplished by differentiation-inducing factors.

26. The method according to claim 22, wherein the culturing conditions comprise maintaining inducing cells in standard media, harvesting the conditioned media, and exposing CNS stem cells to the conditioned media containing soluble stimulants secreted by the inducing cells.

27. The method according to claim 26, wherein the stimulants are isolated from the conditioned media.

28. The method according to claim 22, wherein the contacting is accomplished by co-culturing with organ-specific inducing cell types.

29. The method according to claim 22, wherein the deter g is made by quantitative reverse transcriptase-polymerase chain reaction (QRT-PCR).

30. The method according to claim 22, wherein the determination is made by immunocytochemical characterization of the expression of cell-specific markers.

31. The method according to claim 22, wherein the cell-specific markers are members of the group consisting essentially of nestin, MAP-2, GFAP, Lhx-3, Pit-1, prolactin, Isl-1, insulin, GATA-4, myosin and troponin IC, and wherein the presence of nestin indicates stem cell properties, the presence of MAP-2 indicates differentiation into neuronal cells, the presence of GFAP indicates differentiation into glial cells, the presence of transcription factors Lhx-3 and/or Pit-1 and/or the hormones hGH and Prl indicate differentiation into pituitary cells, the presence of GATA-4, myosin, and/or troponin IC indicate differentiation into pulsatile cardiac cells, and the presence of Isl-1 and/or insulin indicate differentiation into pancreatic cells.

32. A method for treating a subject by populating and/or repopulating cells in depleted or defective organs and/or tissues with pluripotent CNS stem cells induced in vivo or in vitro to specifically differentiate into functional cell types of the affected organ or tissues, comprising:
inducing trans-differentiation of pluripotent CNS stem cells into various other cell types by
harvesting pluripotent stem cells from CNS tissue;
placing the harvested cells into cell culture,
culturing the cells under conditions suitable for maintaining their pluripotency,
contacting the cultured pluripotent cells in vitro or in vivo with differentiation-inducing factors;
determining presence of differentiation into a particular cell type by characterizing expression of cell-specific properties; and
introducing these differentiated cell types to populate and/or repopulate defective areas of said tissues and/or organs.

33. The method according to claim 32, wherein the differentiation-inducing factors are soluble.

34. The method according to claim 32, wherein the source of differentiation-inducing factors are cells in co-culture or the cells of said subject in vivo.

35. The method according to claim 32, wherein the populating and/or repopulating is accomplished by a member of the group including grafting, gene therapy, factor delivery, tissue engineering and organ development.

36. The method according to claim 32, wherein the differentiated CNS cells are used as a conduit for gene therapy or factor delivery to prevent or treat disease.

37. A method for identifying functionality of certain genes, proteins and regulation in various organ and tissue cell types useful in gene discovery, drug discovery, elucidation of differentiation pathways, genetic markers, regulatory factors and biological regulation, comprising:
inducing bans-differentiation of pluripotent central nervous system stem cells into various other cell types by
harvesting the pluripotent stem cells from tissues and organs,
placing the harvested cells into cell culture,
culturing the cells under conditions suitable for maintaining their pluripotency,
contacting the cultured pluripotent cells with differentiation-inducing soluble factors or differentiated cells;
determining the differentiation into a particular cell type by characterizing expression cell-specific properties; and
using these cell types to identify involvement of genes, efficacy of drugs, differentiation pathways, genetic markers and regulatory factors and biological regulation.

38. The method according to claim ?37, wherein the differentiated CNS cells can be used to produce biological factors such as hormones and other vital proteins.

39. A method for isolating and identifying soluble differentiation-inducing factors capable of inducing differentiation of pluripotent central nervous system stem cells into various other cell Apes, comprising:
placing differentiation-inducing cells into cell culture;
culturing the cells under conditions suitable for maintaining their integrity,
harvesting partially spent and conditioned culture medium;
fractionating the conditioned medium;
contacting pluripotent stem cells with the fractions in cell culture;
determining differentiation-inducing effectiveness of each fraction by characterizing expression of cell-specific properties acquired by the induced stem cells to identify the fraction comprising differentiation-inducing factor or factors;
isolating the factor, and
identifying the molecular composition of the factor.

40. The method according to claim 39, wherein the isolated factors are produced in quantity to provide available resources for differentiating pluripotent cells from autologous, homologous, heterologous, or stem cell line sources.

41. The method according to claim 40, wherein the production is by chemical means.

42. The method according to claim 40, wherein the production is by genetic expression.

43. The method according to claim 42, wherein the expression is a natural occurrence in certain cell types.

44. The method according to claim 42, wherein the expression is induced by gene insertion.

45. The method according to claim 44, wherein the gene is inserted into pluripotent stem cells, which cells are capable of proliferation and expression of large amounts of said factors.

46. The method according to claim 44, wherein the gene is inserted into the gene pool of other organisms suitable for expression and recovery of large amounts of said factors.

47. The method according to claim 44, wherein the gene insertion is by methods known to those accomplished in the field.

48. The method according to claim 39, wherein the isolated factor is used to stimulate pluripotent stem cells into directed differentiation in the absence of inducing cell types.
wherein the inducing cells are unavailable for co-culture, or are depleted or defective in a subject.

49. The method according to claim 48, wherein the stimulation is in vitro or in vivo.

50. The method according to claim 49, wherein the in vivo stimulation is accomplished by contacting a subject's cells with the isolated factor.

51. The method according to claim 50, wherein the contacting is by injection or infusion, or other means known to those in the field of administering drugs to subjects.

52. A pharmaceutical composition, comprising:
an effective amount of a differention-inducing factor in a pharmaceutically acceptable carrier.
